(19) United States
(12) Patent Application Publication
(10) Pub. No.: US 2018/0250054 A1
(43) Pub. Date: Sep. 6, 2018

(54) ULTRASOUND TRANSUCER WITH ELECTRODE FEELERS

(71) Applicant: PYTHAGORAS MEDICAL LTD., Herzliya (IL)

(72) Inventors: Yossi GROSS, Moshav Mazor (IL); Yehuda ZADOK, Holon (IL); Ronen NEEMAN, Givatayim (IL)

(73) Assignee: PYTHAGORAS MEDICAL LTD., Herzliya (IL)

(21) Appl. No.: 15/450,591

(22) Filed: Mar. 6, 2017

(51) Int. Cl. A61B 18/00; A61B 5/0215; A61B 18/14

(52) U.S. Cl. CPC A61B 18/00; A61B 5/0215; A61B 18/1492; A61B 2018/0022; A61B 2018/00267; A61B 2018/00994; A61B 2018/00434; A61B 2018/00511; A61B 2018/00577; A61B 2018/00642; A61B 2018/00958; A61B 2018/00404

(57) ABSTRACT

Apparatus is provided, comprising: (a) an elongate member, having a proximal portion and a distal portion; (b) an extracorporeal controller, coupled to the proximal portion of the elongate member, and comprising a user interface and control circuitry; and (c) an effector unit. The effector unit is coupled to the distal portion of the elongate member, defines a longitudinal axis, and has a contracted state and an expanded state. The effector unit comprises a plurality of electrodes, operable by the control circuitry, and disposed circumferentially around the central longitudinal axis of the effector unit; and an ultrasound transducer, operable by the control circuitry. In the expanded state, each electrode is disposed further radially outward from (i) the central longitudinal axis of the effector unit, and (ii) the transducer compared to the contracted state, and the transducer is oriented to direct ultrasound radially outwardly.

### FIELD OF THE INVENTION

Applications of the present invention relate generally to electrical manipulation of nerve tissue. Some applications of the present invention relate more specifically to apparatus and techniques for facilitating location, excitation, and/or ablation of nerve tissue.

### BACKGROUND

Hypertension is a prevalent condition in the general population, particularly in older individuals. Sympathetic nervous pathways, such as those involving the renal nerve, are known to play a role in regulating blood pressure. Ablation of renal nerve tissue from the renal artery is a known technique for treating hypertension.

### SUMMARY OF THE INVENTION

Apparatus is provided in which an effector unit has an ultrasound transducer and electrodes. The effector unit is advanced into a blood vessel of a subject. The effector unit has an expanded state in which it presses the electrodes into the vessel wall of the blood vessel, thereby holding the ultrasound transducer in a position and orientation suitable for the application of ablative ultrasound to the vessel wall. A non-ablative current (e.g., an excitatory current or a blocking current) is applied to the vessel wall in order to facilitate location of nerve tissue within the vessel wall, and/or monitoring of the progress of ablation of the nerve tissue.

There is therefore provided, in accordance with an application of the present invention, apparatus for use with a blood vessel of a subject, the blood vessel having a vessel wall, and the apparatus including:
an elongate member, having a proximal portion, and a distal portion that defines a central longitudinal axis and is advanceable through the blood vessel;
an extracorporeal controller, coupled to the proximal portion of the elongate member, and including a user interface and control circuitry; and
an effector unit:
   having a proximal end and a distal end, and defining a central longitudinal axis therebetween,
   being coupled to the distal portion of the elongate member,
   having a contracted state and an expanded state, and including:
      a plurality of electrodes, operable by the control circuitry, and disposed circumferentially around the central longitudinal axis of the effector unit; and
      an ultrasound transducer, operable by the control circuitry,
         and in the expanded state:
compared to the contracted state, each electrode is disposed further radially outward from (i) the central longitudinal axis of the effector unit, and (ii) the transducer, and
the transducer is oriented to direct ultrasound radially outwardly.

In an application, the effector unit includes an expandable basket, the electrodes are disposed on the expandable basket, and the transducer is disposed inside the expandable basket.

In an application, the effector unit includes a balloon, the electrodes are disposed on the balloon, and the transducer is disposed inside the balloon.

In an application, in the expanded state the electrodes are arranged in a ring around the central longitudinal axis of the effector unit, and the transducer is oriented to direct the ultrasound radially outwardly through the ring.

In an application, the transducer is disposed on the central longitudinal axis of the effector unit.

In an application, the electrodes are distributed circumferentially around the transducer in at least the expanded state of the effector unit.

In an application, the control circuitry is configured to drive the electrodes to apply an excitatory current to the vessel wall.

In an application, the control circuitry is configured to drive the electrodes to apply an ablative current to the vessel wall.

In an application, the apparatus further includes a pressure sensor operable by the control circuitry, and the control circuitry is configured:
while the effector unit is in the expanded state, to operate the electrodes to apply an excitatory current to the vessel wall; and
to receive, from the pressure sensor, data indicative of a change in blood pressure of the subject in response to the excitatory current.

In an application, the control circuitry is configured to, in response to the received data and while the effector unit is in the expanded state, operate the transducer to transmit ultrasound.

In an application, in the expanded state of the effector unit:
the effector unit is dimensioned such that the electrodes press against the vessel wall at a longitudinal site of the vessel, and
the ultrasound transducer is oriented to direct the ultrasound through the vessel wall at the longitudinal site.

There is further provided, in accordance with an application of the present invention, a method, including:
advancing an effector unit into a blood vessel of a subject, the blood vessel having a vessel wall, and the effector unit including:
   a plurality of electrodes disposed circumferentially around a central longitudinal axis of the effector unit, and
   an ultrasound transducer;
securing the transducer in the blood vessel away from the vessel wall by expanding the effector unit into an expanded state such that each electrode of the plurality of electrodes is pressed against the vessel wall; and
while the transducer remains secured in the blood vessel away from the vessel wall, and each electrode remains pressed against the vessel wall, operating the transducer to apply ultrasound through the vessel wall.

In an application, the method further includes, while the transducer remains secured in the blood vessel away from the vessel wall, and each electrode remains pressed against the vessel wall, driving the electrodes to apply an ablative radio-frequency current to the vessel wall.

In an application, the effector unit includes an expandable basket, the electrodes are disposed on the expandable basket, and the transducer is disposed inside the expandable basket, and expanding the effector unit includes expanding the expandable basket.

In an application, the effector unit includes a balloon, the electrodes are disposed on the balloon, and the transducer is disposed inside the balloon, and expanding the effector unit includes inflating the balloon.

In an application, the method further includes, while the transducer remains secured in the blood vessel away from the vessel wall, and each electrode remains pressed against the vessel wall, driving the electrodes to apply an excitatory current to the vessel wall.

In an application, the step of driving the electrodes to apply the excitatory current is performed before the step of operating the transducer to apply ultrasound.

In an application, the step of operating the transducer to apply ultrasound is performed in response to a detected blood pressure change in response to the applied excitatory current.

In an application, the method further includes detecting the blood pressure change.

There is further provided, in accordance with an application of the present invention, apparatus for use with a blood vessel of a subject, the blood vessel having a vessel wall, and the apparatus including:
an elongate member, having a proximal portion, and a distal portion that is advanceable through the blood vessel;
an extracorporeal controller, coupled to the proximal portion of the elongate member, and including a user interface and control circuitry;
an effector unit, coupled to the distal portion of the elongate member, and including:
   an expandable anchor, having a contracted state and an expanded state, and being transitionable therebetween;
   an electrode, operable by the control circuitry, and disposed on the anchor; and
   an ultrasound transducer, operable by the control circuitry,
      and, when the distal portion of the elongate member is disposed in the blood vessel, in the expanded state of the anchor, the anchor:
presses the electrode against the vessel wall at a longitudinal site of the blood vessel, and
holds the transducer away from the vessel wall in an orientation that directs ultrasound transmitted by the transducer toward the electrode.

In an application, the effector unit includes a plurality of electrodes including the electrode.

In an application, the plurality of electrodes is disposed circumferentially around the transducer in at least the expanded state of the expandable anchor.

In an application, the apparatus further includes a pressure sensor operable by the control circuitry, and the control circuitry is configured:
while the anchor is in the expanded state, to operate the electrodes to apply an excitatory current to the vessel wall; and
to receive, from the pressure sensor, data indicative of a change in blood pressure of the subject in response to the excitatory current

In an application, the control circuitry is configured to, in response to the received data and while the anchor is in the expanded state, operate the transducer to transmit ultrasound through the vessel wall at the longitudinal site.

There is further provided, in accordance with an application of the present invention, apparatus for use with a blood vessel of a subject, the blood vessel having a vessel wall, and the apparatus including:
an elongate member, having a proximal portion, and a distal portion that is advanceable through the blood vessel;
an extracorporeal controller, coupled to the proximal portion of the elongate member, and including a user interface and control circuitry;
an effector unit, coupled to the distal portion of the elongate member, and including:
   an expandable anchor, having a contracted state and an expanded state, and being transitionable therebetween;
   an electrode, operable by the control circuitry, and disposed on the anchor; and
   an ultrasound transducer, operable by the control circuitry, and:
in the contracted state, the effector unit defines a contracted distance between the electrode and the transducer; and
in the expanded state:
   the effector unit defines an expanded distance between the electrode and the transducer, the expanded distance being greater than the contracted distance, and
   the transducer is oriented to direct ultrasound toward the electrode.

In an application, the effector unit includes a plurality of electrodes including the electrode.

In an application, the plurality of electrodes is disposed circumferentially around the transducer in at least the expanded state of the expandable anchor.

### BRIEF DESCRIPTION OF THE DRAWINGS

FIGS. 1A-B are schematic illustrations of a system for use with a blood vessel of a subject, in accordance with some applications of the invention;

FIGS. 2A-B are schematic illustrations of an effector unit, in accordance with some applications of the invention;

FIGS. 3A-3B are schematic illustrations of the system being used with a blood vessel of a subject, in accordance with some applications of the invention;

FIGS. 4 and 5 are schematic illustrations of configurations of an ultrasound transducer, in accordance with some applications of the invention; and

FIG. 6 is a schematic illustration of an effector unit, in accordance with some applications of the invention

### DETAILED DESCRIPTION OF EMBODIMENTS

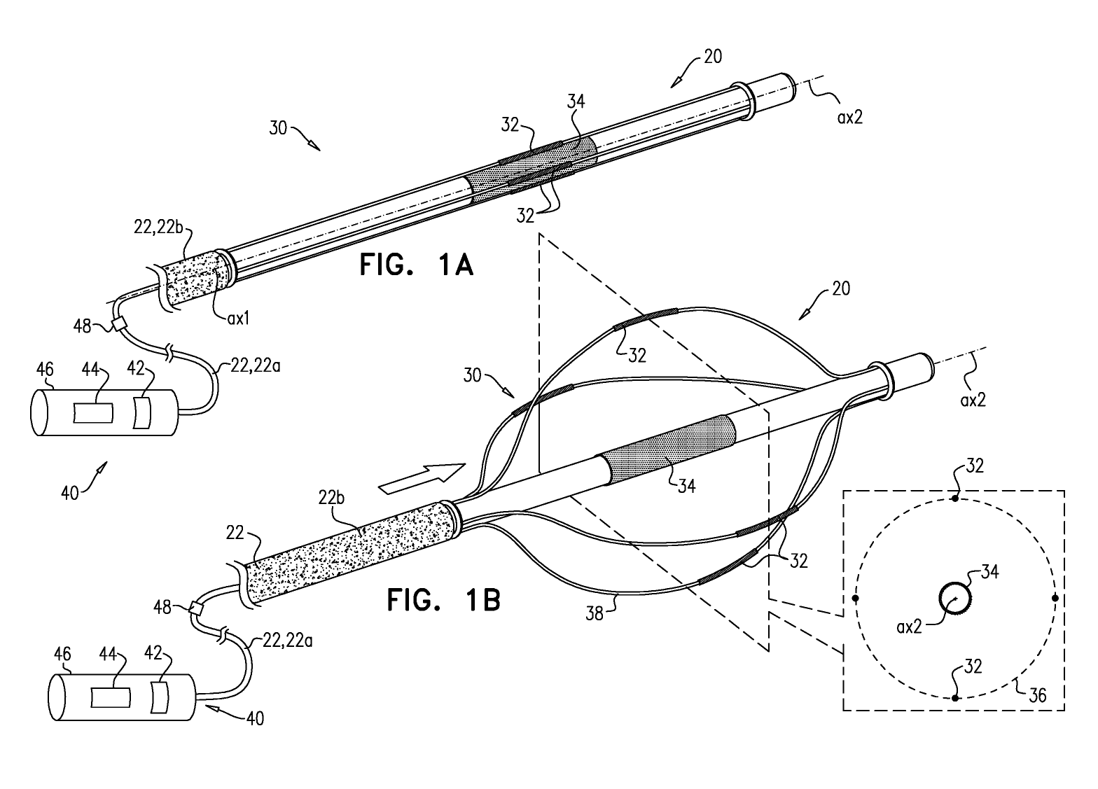

Reference is made to FIGS. 1A-B, which are schematic illustrations of a system 20 for use with a blood vessel of a subject, in accordance with some applications of the invention. System 20 comprises an elongate member (e.g., a shaft or catheter) 22 that has a proximal portion 22a and a distal portion 22b, an effector unit 30 coupled to distal portion 22b, and an extracorporeal controller 40 coupled to proximal portion 22a. Elongate member 22 is flexible, and for some applications actively steerable, so as to facilitate its advancement: to and through the blood vessel. For example, system 20 may be used for renal denervation, in which case elongate member 22 is typically advanced (e.g., transfemorally) through the aorta and into the renal artery. Although elongate member 22 often resides in a non-straight (e.g., tortuous) state, it may nonetheless be described as defining a central longitudinal axis. Similarly, each portion of the elongate member may be described as having a central longitudinal axis that is orthogonal with a transverse cross-section of the elongate member at that portion. For example, and as shown in FIG. 1A, distal portion 22b has a central longitudinal axis ax1.

Extracorporeal controller 40 comprises a user interface 42 and control circuitry 44. User interface 42 may comprise a display and/or control elements such as buttons or knobs. For some applications, and as shown, controller 40 comprises a handle 46 in which control circuitry 44 and/or user interface 42 is disposed.

Effector unit 30 has a proximal end and a distal end, and defines a central longitudinal axis ax2 therebetween. Effector unit 30 is coupled to distal portion 22b of elongate member 22. In many situations, and as shown, axis ax2 of effector unit 30 is collinear with axis ax1 of distal portion 22b. Effector unit 30 has a contracted state (FIG. 1A) and an expanded state (FIG. 1B). Effector unit 30 comprises a plurality of electrodes 32 disposed circumferentially around axis ax2, and an ultrasound transducer 34 that is typically disposed closer than the electrodes to axis ax2 (e.g., on axis ax2). Electrodes 32 and transducer 34 are each operable by control circuitry 44.

In the expanded state of effector unit 30, each electrode is disposed further radially outward from (i) the central longitudinal axis of the effector unit, and (ii) the transducer, compared to when the effector unit is in the contracted state. In the expanded state of effector unit 30 (and typically also in its contracted state), the transducer is oriented to direct ultrasound radially outwardly, i.e., away from axis ax2.

As shown in the cross-section of FIG. 1B, in at least the expanded state electrodes 32 are typically distributed circumferentially around (i.e., surround) axis ax2. That is, if one were to draw connecting lines between the electrodes, axis ax2 (and typically transducer 34) would be disposed within the resulting polygon. In such an arrangement, electrodes 32 may be described as being arranged in a ring 36 around axis ax2, and transducer 34 is oriented to direct ultrasound radially outwardly through the ring. Typically, in at least the expanded state, electrodes 32 are distributed circumferentially around (i.e., surround) transducer 34, i.e., the electrodes longitudinally aligned with the transducer.

Typically, electrodes 32 are distributed circumferentially around axis ax2 also in the contracted state. For some applications, electrodes 32 are distributed circumferentially around (i.e., longitudinally aligned with) transducer 34 also in the contracted state (e.g., as shown in FIG. 1A). For some applications, electrodes 32 are not: longitudinally aligned with transducer 34 in the contracted state, e.g., to reduce the maximum diameter of effector unit 30 in its contracted state.

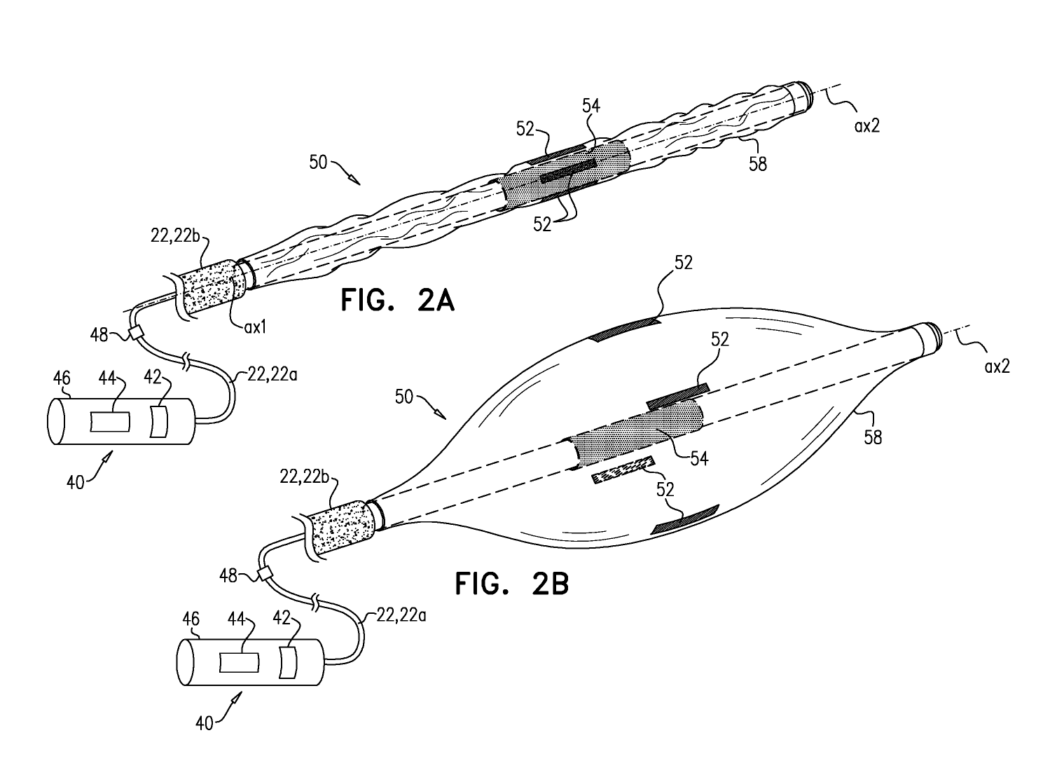

Reference is now also made to FIGS. 2A-B. FIGS. 1A-B show effector unit 30 as comprising an expandable basket 38 (e.g., a plurality of struts that are manipulatable to expand and compress the effector unit) on which electrodes 32 are disposed. For some applications, the effector unit comprises an inflatable balloon, which serves a corresponding function. FIGS. 2A-B are schematic illustrations of an effector unit 50, in accordance with some applications of the invention. Effector unit 50 comprises an inflatable balloon 58, on which electrodes 52 are disposed, and inside which a transducer 54 is disposed. Electrodes 52 and transducer 54 serve functions corresponding to those of electrodes 32 and transducer 54 of effector unit 30, mutatis mutandis. Throughout this patent application, unless stated otherwise, when reference is made to effector unit 30 (or elements thereof), effector unit 50 may be used instead, mutatis mutandis. For simplicity, although balloon 58 is shown as transparent, FIGS. 2A-B do not show wires connecting controller 40 to electrodes 32. Typically, these wires are structurally passive, and it is only the inflation and deflation of balloon 58 that transitions effector unit 50 between its contracted and expanded states.

For some applications, balloon 58 or the fluid with which it is inflated further functions to cool the tissue with which it is in contact. The fluid with which balloon 58 is inflated is substantially acoustically transparent.

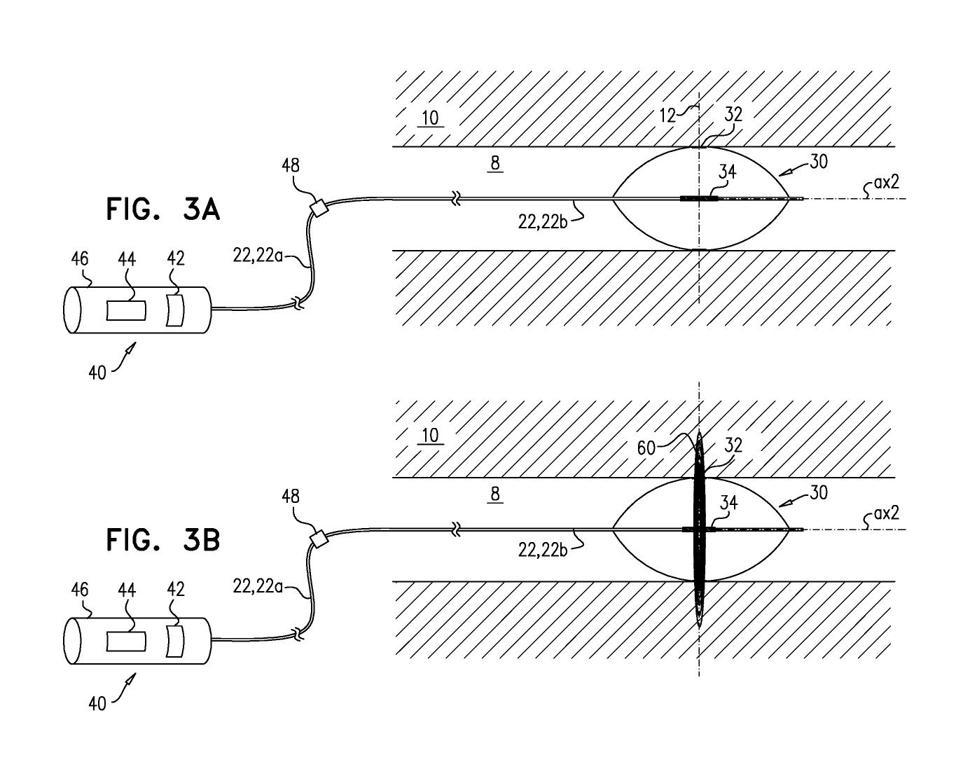

Reference is now made to FIGS. 34-3B, which are schematic illustrations of system 20 being used with a blood vessel (e.g., a renal artery) 8 of a subject, in accordance with some applications of the invention. Distal portion 22b and effector unit 30 are advanced transluminally (e.g., transfemorally) into blood vessel 8, and effector unit 30 is transitioned into its expanded state (FIG. 34). In this state, electrodes 32 are pressed against the vessel wall 10 of the blood vessel at a longitudinal site 12 of the blood vessel. This holds transducer 34 away from vessel wall 10, and orients the transducer such that ultrasound emitted by the transducer is directed toward the electrode, and thereby toward the vessel wall beyond the electrode, e.g., at site 12. This is achieved by the basket design of effector unit 30, or by the balloon design of effector unit 50. Therefore basket 38 and balloon 58 each serves as an expandable anchor.

There is therefore provided, in accordance with some applications of the invention, apparatus comprising:
an elongate member (e.g., member 22), having a proximal portion, and a distal portion that is advanceable through the blood vessel;
an extracorporeal controller (e.g., controller 40), coupled to the proximal portion of the elongate member, and comprising a user interface and control circuitry;
   an effector unit (e.g., unit 30 or unit 50), coupled to the distal portion of the elongate member, and comprising:
   an expandable anchor (e.g., basket 38 or balloon 58), having a contracted state and an expanded state, and being transitionable therebetween;
   an electrode (e.g., electrode 32 or electrode 52), operable by the control circuitry, and disposed on the anchor; and
   an ultrasound transducer (e.g., transducer 34 or transducer 54), operable by the control circuitry,
      wherein, when the distal portion of the elongate member is disposed in the blood vessel, in the expanded state of the anchor, the anchor presses the electrode against the vessel wall at a longitudinal site of the blood vessel, and holds the transducer away from the vessel wall in an appropriate orientation for the application of ultrasound to the vessel wall—e.g., an orientation that directs the ultrasound radially outward, e.g., toward the electrode.

Electrodes 32 are used to locate (longitudinally and/or circumferentially) nerve tissue associated with vessel wall 10 in order to position transducer 34 for application of ablative ultrasound to the nerve tissue. For example, control circuitry 44 may be configured to drive one or more of electrodes 32 to apply excitatory current to vessel wall 10. If the driven electrodes are aligned with and/or close to the nerve tissue, the excitatory current initiates action potentials in the nerve tissue, resulting in a physiological response to the action potentials. The physiological response is therefore indicative that the driven electrodes, and therefore transducer 34, are positioned such that ablative ultrasound emitted by the transducer will be applied in an appropriate location. For some applications, a non-ablative blocking current is used instead of (or in addition to) the excitatory current.

Once an appropriate site has been located, transducer 34 is driven by control circuitry 44 to apply ablative ultrasound 60 to vessel wall 10, typically while electrodes 32 remain in contact with the site on the vessel wall at which they applied the current that identified the site (FIG. 3B). As described hereinabove, basket 38 (i.e., serving as an anchor) presses electrodes 32 against the vessel wall at longitudinal site 12 of the blood vessel, and holds transducer 34 away from the vessel wall in an appropriate orientation for the application of ultrasound to the vessel wall (e.g., an orientation that directs the ultrasound radially outward). This orientation is typically such that the applied ultrasound is directed toward the tissue to which electrodes 32 applied the current—, e.g., toward the electrodes themselves.

For some applications, the excitatory current or non-ablative blocking current may alternatively or additionally be used to monitor the progress of ablation. After application of ablative ultrasound, the current may be (re)applied, and the physiological response (re)measured. A reduction in the physiological response correlates with successful ablation. For some applications, the excitatory current or non-ablative blocking current may be applied simultaneously with the ablative ultrasound, in order to monitor progress of ablation.

In response to this monitoring, the energy level and/or duration of ablation may be adjusted in real-time.

For applications in which system 20 is used for renal denervation, the physiological response that is measured is typically a change in blood pressure of the subject. For some applications, system 20 comprises a pressure sensor 48 (e.g., disposed on elongate member 22) that is operable by control circuitry 44, and the control circuitry is configured to, while the effector unit is in the expanded state, operate the electrodes to apply an excitatory current to the vessel wall, and to receive, from the pressure sensor, data indicative of a change in blood pressure of the subject in response to the excitatory current. For some such applications, control circuitry 44 is configured to, in response to the received data and while the effector unit is in the expanded state, operate (e.g., automatically operate) the transducer to transmit ultrasound 60. Alternatively, controller 40 is manually operated by a user to operate the transducer.

For some applications, the techniques used for using excitatory and/or non-ablative blocking current to locate nerve tissue and monitor progress of ablation are as described (mutatis mutandis) in one or more of the following references, which are incorporated herein by reference:
US 2014/0128865 to Gross;
US 2015/0245867 to Gross; and
WO 2015/170281 to Gross et al.

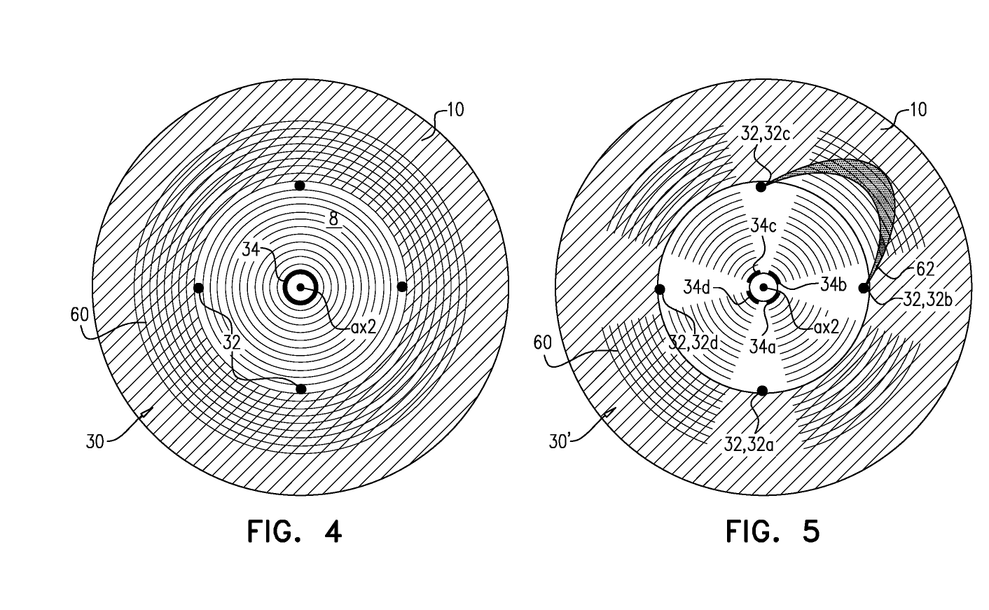

Reference is now made to FIGS. 4 and 5, which are schematic illustrations of configurations of ultrasound transducer 34, in accordance with some applications of the invention. FIG. 4 is a transverse cross-section of vessel 8 and effector unit 30, at longitudinal site 12 of FIG. 3B. Transducer 34 is shown as circumscribing axis ax2, and as being configured to emit ultrasound 60 radially outward from axis ax2 across 360 degrees circumferentially—i.e., in an annular pattern. However, the scope of the invention includes other configurations of the ultrasound transducer. For example, and as shown in FIG. 5, an effector unit 30′ may comprise a plurality of ultrasound transducers 34 (e.g., ultrasound transducers 34a, 34b, 34c, and 34d). As shown in FIG. 5, the plurality of ultrasound transducers may be arranged circumferentially such that each emits ultrasound 60 at a respective different angle. For some applications, and as shown, the transducers are rotationally offset with respect to electrodes 32, such that ultrasound 60 is transmitted primarily between electrodes 32.

In FIG. 5, the four electrodes 32 are given individual reference numerals: 32a, 32b, 32c, and 32d.

For some applications, the excitatory or non-ablative blocking current that is used to locate nerve tissue and/or monitor ablation progress is applied between adjacent electrodes 32. For such applications, the current therefore passes through the sector of vessel wall 10 disposed between these two electrodes, and may reach its maximum depth within the vessel wall midway along this sector. For example, in FIG. 5 an excitatory current 62 is shown being applied between electrodes 32b and 32c, and therefore passing through the sector of vessel wall 10 that is disposed between these two electrodes. Ultrasound transducer 34b is directed toward this sector, and therefore when driven it emits ultrasound toward this sector.

It is hypothesized by the inventors that a configuration such as that shown in FIG. 5 may advantageously facilitate locating of nerve tissue circumferentially (rather than just longitudinally), and specifically targeting ablative ultrasound toward this located nerve tissue.

For some applications, when effector unit 30′ is transitioned into its contracted state, electrodes 32 become disposed in the space circumferentially between transducers 34 (i.e., the electrodes intercalate with the transducers), such that a greatest diameter of the effector unit is smaller than a corresponding effector unit that has a fully circumferential transducer (e.g., effector unit 30). Therefore, for some applications, a method is provided in which an effector unit is transitioned from its contracted state into its expanded state by moving electrodes 32 radially outward from between transducers 34. For such applications, the user may also transition the effector unit from its expanded state into its contracted state by moving electrodes 32 radially inward to between transducers 34 (e.g., prior to introducing the effector unit into the body of the subject, or prior to withdrawal of the effector unit after treatment of the subject).

It is to be noted that although FIG. 5 shows ultrasound transducers 34a-d as each having an arc-shaped transverse cross-section, the scope of the invention includes the transducers having being other shapes, such as having a flat (e.g., rectangular) transverse cross-section.

As described hereinabove, although the figures of this patent application show the effector units as comprising four electrodes, they may comprise fewer (e.g., 1, 2, or 3) or more (e.g., 6, 8, or 12) electrodes. It is also to be noted that effector units such as effector unit 30′ may comprise fewer (e.g., 1, 2, or 3) or more (e.g., 6, 8, or 12) ultrasound transducers. For example, it is hypothesized by the inventors that for some applications it is advantageous for effector unit 30′ to comprise three electrodes and three transducers.

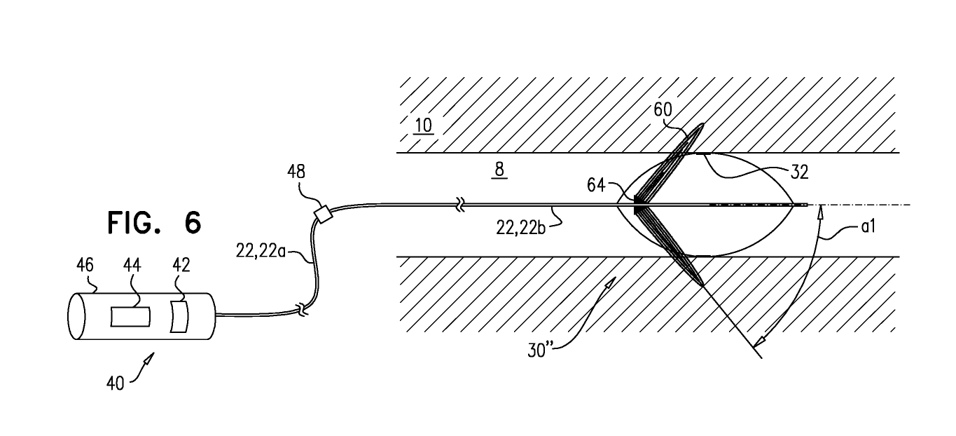

Reference is now made to FIG. 6, which is a schematic illustration of an effector unit 30″, in accordance with some applications of the invention. FIG. 3B shows ultrasound 60 being transmitted at angles that are perpendicular to axis ax2. For some applications in which the angles are perpendicular to axis ax2, and as shown in FIG. 3B, in at least the expanded state electrodes 32 are disposed circumferentially around transducer 34 (i.e., the transducer is longitudinally aligned with the electrodes). For such applications, the transducers are thereby oriented to direct ultrasound 60 toward and past electrodes 32.

However, for some applications the transducer (e.g., a transducer 64) is oriented to transmit ultrasound 60 radially outward from axis ax2 at an angle alpha_1 that is less than 90 degrees from axis ax2. For some such applications, in at least the expanded state electrodes 32 are disposed distally (as shown in FIG. 6) or proximally from the transducer (i.e., the transducer is not longitudinally aligned with the electrodes). For some such applications, the transducers are oriented to direct ultrasound 60 past electrodes 32 but not necessarily toward the electrodes, e.g., to target tissue behind the electrode, as shown in FIG. 6.

FIGS. 1A-B show transducer 34 as generally cylindrical, i.e., the transducer surface is generally parallel with axis ax2. However, it is to be understood that the scope of the invention includes other transducer shapes. For example, the transducer may be conical (as shown in FIG. 6). For some applications, ultrasound 60 is not focused. For some applications, ultrasound 60 is focused ultrasound (e.g., low intensity focused ultrasound (LIFU) or high intensity focused ultrasound (HIFU)). For some applications, the transducer is shaped to focus the ultrasound. For example, rather than a cylindrical transducer the transducer may be hourglass shaped, or rather than a conical transducer the transducer may be funnel shaped.

Effector units 30 and 50 are shown as comprising a of four electrodes, but they may comprise fewer (e.g., 1, 2, or 3 electrodes) or more (e.g., 6, 8, or 12) electrodes. Effector units 30 and 50 are shown as comprising a single “ring” of electrodes, but they may alternatively comprise more than one ring of electrodes.

For some applications, ablative current (e.g., radio frequency (RF) current) may also be applied to the vessel wall, in order to ablate the nerve tissue. For some applications, the ablative current is applied by control circuitry driving electrodes 32 to apply the ablative current. For some applications, additional electrodes are provided on the effector unit for the application of the ablative current. For some applications, the ablative current is applied simultaneously with the ablative ultrasound. For some applications, the modality or modalities used is/are dependent on the depth of the nerve tissue within the vessel wall. For example, RF current may be used to ablate shallower nerve tissue, and ultrasound may be used to ablate deeper nerve tissue. Determination of the depth of nerve tissue to be ablated may be achieved using techniques described in one or more of the following references, which are incorporated herein by reference:
US 2014/0128865 to Gross;
US 2015/0245867 to Gross; and
WO 2015/170281 to Gross et al.

As used in the present application, including in the claims, a “central longitudinal axis” of an elongate structure (or portion thereof) is the set of all centroids of transverse cross-sectional sections of the structure along the structure (or portion thereof). Thus the cross-sectional sections are locally orthogonal to the central longitudinal axis, which runs along the structure (or portion thereof). (For a structure that is circular in cross-section, the centroids correspond with the centers of the circular cross-sectional sections.)

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a blood vessel of a subject, the blood vessel having a vessel wall, and the apparatus comprising:
an elongate member, having a proximal portion, and a distal portion that defines a central longitudinal axis and is advanceable through the blood vessel;
an extracorporeal controller, coupled to the proximal portion of the elongate member, and comprising a user interface and control circuitry; and
an effector unit:
having a proximal end and a distal end, and defining a central longitudinal axis therebetween,
being coupled to the distal portion of the elongate member,
having a contracted state and an expanded state, and comprising:
a plurality of electrodes, operable by the control circuitry, and disposed circumferentially around the central longitudinal axis of the effector unit; and
an ultrasound transducer, operable by the control circuitry,
wherein in the expanded state:
compared to the contracted state, each electrode is disposed further radially outward from (i) the central longitudinal axis of the effector unit, and (ii) the transducer, and
the transducer is oriented to direct ultrasound radially outwardly.

2. The apparatus according to claim 1, wherein the effector unit comprises an expandable basket, the electrodes are disposed on the expandable basket, and the transducer is disposed inside the expandable basket.

3. The apparatus according to claim 1, wherein the effector unit comprises a balloon, the electrodes are disposed on the balloon, and the transducer is disposed inside the balloon.

4. The apparatus according to claim 1, wherein in the expanded state the electrodes are arranged in a ring around the central longitudinal axis of the effector unit, and the transducer is oriented to direct the ultrasound radially outwardly through the ring.

5. The apparatus according to claim 1, wherein the transducer is disposed on the central longitudinal axis of the effector unit.

6. The apparatus according to claim 1, wherein the electrodes are distributed circumferentially around the transducer in at least the expanded state of the effector unit.

7. The apparatus according to claim 1, wherein the control circuitry is configured to drive the electrodes to apply an excitatory current to the vessel wall.

8. The apparatus according to claim 1, wherein the control circuitry is configured to drive the electrodes to apply an ablative current to the vessel wall.

9. The apparatus according to claim 1, further comprising a pressure sensor operable by the control circuitry, wherein the control circuitry is configured:
while the effector unit is in the expanded state, to operate the electrodes to apply an excitatory current to the vessel wall; and
to receive, from the pressure sensor, data indicative of a change in blood pressure of the subject in response to the excitatory current.

10. The apparatus according to claim 9, wherein the control circuitry is configured to, in response to the received data and while the effector unit is in the expanded state, operate the transducer to transmit ultrasound.

11. The apparatus according to claim 10, wherein, in the expanded state of the effector unit:
the effector unit is dimensioned such that the electrodes press against the vessel wall at a longitudinal site of the vessel, and
the ultrasound transducer is oriented to direct the ultrasound through the vessel wall at the longitudinal site.

12-19. (canceled)

20. Apparatus for use with a blood vessel of a subject, the blood vessel having a vessel wall, and the apparatus comprising:
an elongate member, having a proximal portion, and a distal portion that is advanceable through the blood vessel;
an extracorporeal controller, coupled to the proximal portion of the elongate member, and comprising a user interface and control circuitry;
an effector unit, coupled to the distal portion of the elongate member, and comprising:
an expandable anchor, having a contracted state and an expanded state, and being transitionable therebetween;
an electrode, operable by the control circuitry, and disposed on the anchor; and
an ultrasound transducer, operable by the control circuitry,
wherein, when the distal portion of the elongate member is disposed in the blood vessel, in the expanded state of the anchor, the anchor:
presses the electrode against the vessel wall at a longitudinal site of the blood vessel, and
holds the transducer away from the vessel wall in an orientation that directs ultrasound transmitted by the transducer toward the electrode.

21. The apparatus according to claim 20, wherein the effector unit comprises a plurality of electrodes including the electrode.

22. The apparatus according to claim 21, wherein the plurality of electrodes is disposed circumferentially around the transducer in at least the expanded state of the expandable anchor.

23. The apparatus according to claim 20, further comprising a pressure sensor operable by the control circuitry, wherein the control circuitry is configured:
while the anchor is in the expanded state, to operate the electrode to apply an excitatory current to the vessel wall; and
to receive, from the pressure sensor, data indicative of a change in blood pressure of the subject in response to the excitatory current

24. The apparatus according to claim 23, wherein the control circuitry is configured to, in response to the received data and while the anchor is in the expanded state, operate the transducer to transmit ultrasound through the vessel wall at the longitudinal site.

25. Apparatus for use with a blood vessel of a subject, the blood vessel having a vessel wall, and the apparatus comprising:
an elongate member, having a proximal portion, and a distal portion that is advanceable through the blood vessel;
an extracorporeal controller, coupled to the proximal portion of the elongate member, and comprising a user interface and control circuitry;
an effector unit, coupled to the distal portion of the elongate member, and comprising:
an expandable anchor, having a contracted state and an expanded state, and being transitionable therebetween;
an electrode, operable by the control circuitry, and disposed on the anchor; and
an ultrasound transducer, operable by the control circuitry,
wherein:
in the contracted state, the effector unit defines a contracted distance between the electrode and the transducer; and
in the expanded state:
the effector unit defines an expanded distance between the electrode and the transducer, the expanded distance being greater than the contracted distance, and
the transducer is oriented to direct ultrasound toward the electrode.

26. The apparatus according to claim 25, wherein the effector unit comprises a plurality of electrodes including the electrode.

27. The apparatus according to claim 26, wherein the plurality of electrodes is disposed circumferentially around the transducer in at least the expanded state of the expandable anchor.
